(19) United States
(12) Patent Application Publication
(10) Pub. No.: US 2022/0163500 A1
(43) Pub. Date: May 26, 2022

(54) INDEX MATERIAL FOR CLASSIFICATION OF KOREAN GINSENG AND CLASSIFICATION OF KOREAN GINSENG SPECIES USING THE SAME

(71) Applicant: FAMENITY CO., LTD., Uiwang-si (KR)

(72) Inventors: Ji Won LEE, Gwacheon-si (KR); Sung Su KIM, Gwacheon-si (KR); Jun Kee HONG, Yongin-si (KR); Ji Won MOON, Chungju-si (KR)

(21) Appl. No.: 17/531,487

(22) Filed: Nov. 19, 2021

(30) Foreign Application Priority Data
Nov. 20, 2020 (KR) 10-2020-0157159

(51) Int. Cl. G01N 33/02; G01N 30/86; G01N 30/72; G01N 30/78; G01N 30/06; G01N 30/74

(52) U.S. Cl. CPC G01N 33/025; G01N 30/8631; G01N 30/72; G01N 2030/884; G01N 30/78; G01N 30/06; G01N 30/74; G01N 30/8679

(57) ABSTRACT

The present disclosure relates to a marker for identifying Korean ginseng cultivars and a method of identifying Korean ginseng cultivars using the same. Specifically, the present disclosure provides a marker for identification of Korean ginseng cultivars, which is obtained by identifying and separating an active ingredient, which is contained only in Korean ginseng cultivars, among non-saponin components. In addition, the present disclosure makes it possible to determine whether a product sold as Korean ginseng is Korean ginseng by separating and purifying the active ingredient without using a conventional molecular biological method.

### CROSS-REFERENCE TO RELATED APPLICATION

This application claims priority to Korean Patent Application No. 10-2020-0157159, filed on Nov. 20, 2020, the disclosure of which is incorporated herein by reference in its entirety.

### BACKGROUND

### Technical Field

The present disclosure relates to a marker for identifying Korean ginseng cultivars and a method of identifying Korean ginseng cultivars using the same, and more particularly, to a marker for identifying Korean ginseng cultivars using an active ingredient, which is contained only in Korean ginseng and is not contained in a plurality of other ginseng species, and a method of identifying Korean ginseng cultivars using the same.

### Description of the Related Art

Ginseng (Panax ginseng Meyer) is one of the major oriental medicinal plants and has been used for a long time as a primary treatment for relieving fatigue and weakness. This plant, which grows naturally in Korea and northeastern China, is commonly called Korean ginseng and is currently cultivated around the world, particularly in Northeast Asia.

As a pharmacological component of ginseng, an amorphous substance which is a kind of saponin mixture was separated from the root of American ginseng in 1854. Since then, scientific research on the active ingredient of ginseng has begun. In 1957, Brekhman, a Soviet scientist, emphasized that saponin glycoside is an active ingredient of ginseng, and since then, research on the chemical active ingredients of ginseng has started in earnest, and intensive research on ginseng saponins were conducted. In the 1960s, many studies on the active ingredients of ginseng were actively conducted in the field of biochemistry.

In most ginseng species, various active ingredients, including ginsenosides, polysaccharides, peptides, polyacetylenic alcohols and fatty acids, are found.

Modern science revealed that Korean ginseng has pharmacological effects, including brain function improvement, pain reduction effect, cancer prevention effect including anti-tumor activity, immune function improvement, anti-diabetic effect, liver function improvement, blood pressure control, anti-fatigue and anti-stress effects, alleviation of menopausal disorders, sexual function improvement, and antioxidant and anti-aging effects.

For this reason, Korean ginseng has become the most popular medicinal plant worldwide.

Ginseng is called by various names depending on the origin and cultivation. The Panax genus consists of 17 species, of which Korean ginseng (P. ginseng C.A. Meyer), Chinese ginseng (P. notoginseng), and American ginseng (P. quinquefolium) are known for their medicinal effects.

Korean ginseng is broadly divided into three types: Jakyung, Chungkyung, and Hwangsook. To date, a total of 12 Korean ginseng cultivars have been bred from three types of pure lines by the pure-line selection method. Specifically, the Jakyung type includes Yunpoong, Gopoong, Sunpoong, Sunwun, Sunweon, Sunhyang, K-1, Chunil, and Chunang, and the Chungkyung type includes Chunpoong and Chungsun, and the Hwangsook type includes Gumpoong.

As molecular biological methods used for identification of ginseng species so far, several approaches have been attempted, including random amplified polymorphic DNA (RAPD), inter-simple sequence repeats (ISSR), restriction fragment length polymorphism (RFLPs), and simple sequence repeats (SSR).

However, these methods lack reproducibility and reliability. Therefore, there is a need to quickly prevent an act that disrupts the market order by selling other ginseng species passed off as Korean ginseng at high prices.

[Prior Art Documents]

[Patent Documents]

(Patent Document 1) KR 10-1481723 B1

### SUMMARY

An object of the present disclosure is to provide a marker for identifying Korean ginseng cultivars and a method of identifying Korean ginseng cultivars using the same.

Another object of the present disclosure is to provide a marker for identification of Korean ginseng cultivars, which is obtained by identifying and separating an active ingredient, which is contained only in Korean ginseng cultivars, among non-saponin components.

Still another object of the present disclosure is to provide a method for identifying Korean ginseng cultivars, which is capable of determining whether a product sold as Korean ginseng is Korean ginseng by separating and purifying the active ingredient without using a conventional molecular biological method.

To achieve the above objects, the present disclosure provides a marker for identifying Korean ginseng cultivars, which is an active ingredient contained in a fraction separated from an extract of Korean ginseng (Panax ginseng C. A. Meyer), wherein the active ingredient is a non-saponin component, and the active ingredient shows a first peak at 13.3 min and a second peak at 13.5 min when the UPLC spectrum of the fraction is measured using a PAD detector at a UV wavelength of 254 nm.

The active ingredient may be a compound represented by the following Formula 1:

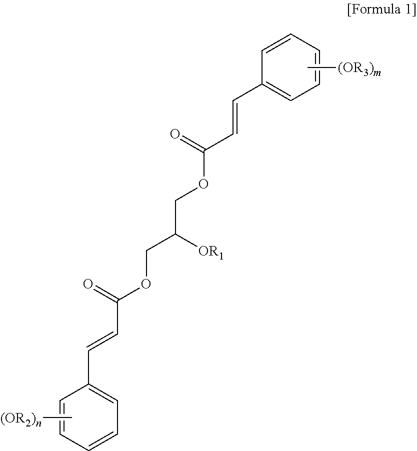

wherein

n and m are the same as or different from each other and are each independently an integer ranging from 1 to 5, and

R₁ to R₃ are the same as or different from one another and are each independently selected from the group consisting of hydrogen, hydrogen, deuterium, a ketone group, an alkyl group having 1 to 30 carbon atoms, and an alkenyl group having 2 to 10 carbon atoms.

The active ingredient represented by Formula 1 may have a molar mass of 410 to 430 g/mol.

The active ingredient may be a compound represented by the following Formula 2:

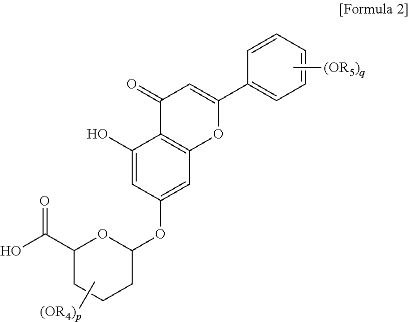

wherein

p is an integer ranging from 1 to 3,

q is an integer ranging from 1 to 5, and

R₄ and R₅ are the same as or different from each other and are each independently selected from the group consisting of hydrogen, deuterium, a ketone group, an alkyl group having 1 to 30 carbon atoms, and an alkenyl group having 2 to 10 carbon atoms.

The active ingredient represented by Formula 2 may have a molar mass of 455 to 465 g/mol.

The extract of Korean ginseng may be obtained by extraction using 70% ethanol as an extraction solvent.

The fraction may be obtained by fractionating a 70% ethanol extract of Korean ginseng using methanol as a fractionation solvent.

The present disclosure also provides a method of identifying Korean ginseng cultivars using the marker, the method including steps of: 1) separating ginseng species samples from different ginseng groups whose species are unidentifiable; 2) preparing ginseng extracts by powdering each of the separated ginseng species samples, followed by extraction using an extraction solvent; 3) obtaining fractions by fractionation of the ginseng extracts; and 4) measuring the UPLC spectrum of the fraction for each ginseng species using a PAD detector at a UV wavelength of 254 nm, wherein ginseng containing an active ingredient showing a first peak at 13.3 min and a second peak at 13.5 min in the results of measuring the UPLC spectrum is Korean ginseng (Panax ginseng C.A. Meyer).

Hereinafter, the present disclosure will be described in more detail.

A marker for identifying Korean ginseng cultivars according to one embodiment of the present disclosure is an active ingredient contained in a fraction separated from an extract of Korean ginseng (Panax ginseng C.A. Meyer), wherein the active ingredient is a non-saponin component, and the active ingredient shows a first peak at 13.3 min and a second peak at 13.5 min when the UPLC spectrum of the fraction is measured using a PAD detector at a UV wavelength of 254 nm.

Ginseng is registered in herbal medicine, and thus it can be seen that ginseng has been consumed as a medicinal plant for a long time. It was found that the reason why ginseng can be used as a medicinal plant is because of the action of substances called ginseng saponins. The saponin components present in ginseng were specifically named ginsenosides.

Since studies on these ginsenosides have been actively conducted, analysis of saponin components in ginseng of all origins has been reported, but it is difficult to estimate the origin of saponins through this analysis.

Therefore, in the present specification, several ginseng species of domestic and foreign origin were selected, and non-saponin components capable of distinguishing between these ginseng species were identified, and in particular, a marker capable of identifying Korean ginseng (Panax ginseng CA Meyer) cultivars was identified.

The active ingredient may be a non-saponin component, and the active ingredient may show a first peak at 13.3 min and a second peak at 13.5 min when the UPLC spectrum of the fraction is measured using a PAD detector at a UV wavelength of 254 nm.

Specifically, the active ingredient is a compound represented by the following Formula 1, and has a molar mass of 410 to 430 g/mol, preferably 426.4 g/mol:

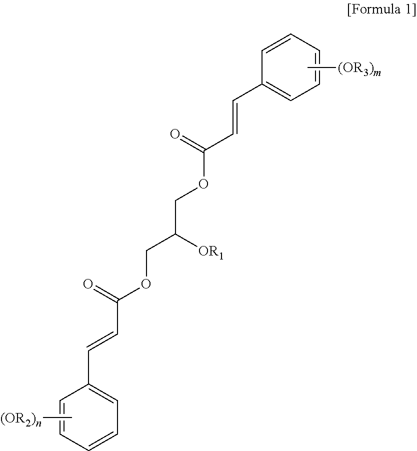

wherein

n and m are the same as or different from each other and are each independently an integer ranging from 1 to 5, and

R₁ to R₃ are the same as or different from one another and are each independently selected from the group consisting of hydrogen, hydrogen, deuterium, a ketone group, an alkyl group having 1 to 30 carbon atoms, and an alkenyl group having 2 to 10 carbon atoms.

—OR₂ and —OR₃ of the compound represented by Formula 1 are —OH groups, and —OR₁ thereof is a —OCOOH group, but they are not limited to the above examples.

More preferably, the compound represented by Formula 1 may be a compound represented by the following Formula 3:

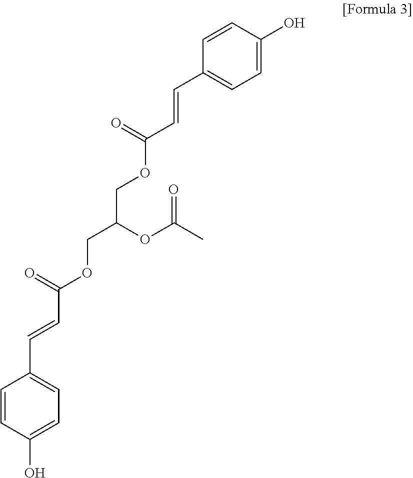 [SMILES: CC(=O)OC(COC(=O)/C=C/c1ccc(O)cc1)COC(=O)/C=C/c1ccc(O)cc1]

In another embodiment, the active ingredient may be a compound represented by the following Formula 2 and has a molar mass of 455 to 465 g/mol, preferably 462.4 g/mol:

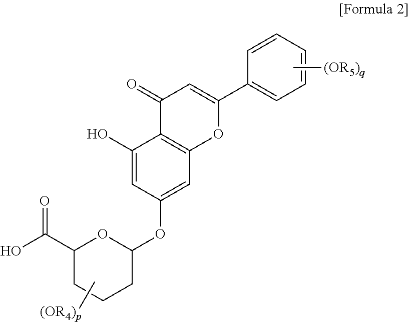

wherein

p is an integer ranging from 1 to 3,

q is an integer ranging from 1 to 5, and

R₄ and R₅ are the same as or different from each other and are each independently selected from the group consisting of hydrogen, deuterium, a ketone group, an alkyl group having 1 to 30 carbon atoms, and an alkenyl group having 2 to 10 carbon atoms.

Preferably, the compound represented by Formula 2 may be a compound represented by the following Formula 4:

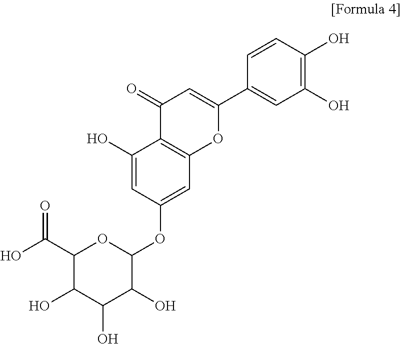 [SMILES: O=C(O)C1OC(Oc2cc(O)c3c(=O)cc(-c4ccc(O)c(O)c4)oc3c2)C(O)C(O)C1O]

The extract of Korean ginseng may be prepared by a conventional method for preparing an extract.

Specifically, the extract of Korean ginseng may be obtained by a method including steps of: powdering Korean ginseng; leaching the Korean ginseng powder with an organic solvent to obtain a sample; drying the sample; re-leaching the dried sample with an organic solvent; drying the sample; leaching the dried sample using water; and leaching.

The extract obtained by extraction with the organic solvent may be further subjected to a fractionation step using an organic solvent.

The method for preparing the extract may be an extraction method known in the art, such as an ultrasonic extraction method, a leaching method or a reflux extraction method. Specifically, the extract may be an extract obtained by washing and drying ginseng to remove foreign matter and then extracting the ginseng with water, an alcohol having 1 to 6 carbon atoms, or a mixed solvent thereof. Alternatively, the extract may also be an extract obtained by sequentially applying the solvents to the sample.

The reflux extraction method is performed by refluxing 10 to 30 g of the pulverized natural product in 100 mL of a 50 to 100% alcohol having 1 to 6 carbon atoms for 1 to 3 hours. More specifically, the reflux extraction method is performed by refluxing 10 to 20 g of the powdered natural product in 100 mL of a 70 to 90% alcohol having 1 to 4 carbon atoms for 1 to 2 hours.

The leaching method is performed by leaching using a 50 to 100% alcohol having 1 to 6 carbon atoms as an extraction solvent at 15 to 30° C. for 24 to 72 hours. More specifically, the leaching method is performed by leaching using a 70 to 80% alcohol having 1 to 6 carbon atoms as an extraction solvent at 20 to 25° C. for 30 to 54 hours.

The ultrasonic extraction method is performed by extraction using a 50 to 100% alcohol having 1 to 6 carbon atoms as an extraction solvent at 30 to 50° C. for 0.5 to 2.5 hours. Specifically, the ultrasonic extraction method includes performing extraction using a 70 to 80% alcohol having 1 to 6 carbon atoms as an extraction solvent at 40 to 50° C. for 1 to 2.5 hours.

The extraction solvent may be used in an amount equal to 2 to 50 times, more specifically 2 to 20 times, the weight of the sample. For leaching and extraction, the sample may be left to stand in the extraction solvent for 1 to 72 hours, more specifically 24 to 48 hours.

After extraction, the extract may be fractionated sequentially using fresh fractionation solvents. The fractionation solvent that is used for fractionation of the extract is any one or more selected from the group consisting of water, hexane, butanol, ethyl acetic acid, ethyl acetate, methylene chloride, and mixtures thereof. Preferably, the fractionation solvent is ethyl acetate or methylene chloride.

The obtained extract or fraction may also be concentrated or freeze-dried.

Specifically, the extract of Korean ginseng may be obtained by extraction using 70% ethanol as an extraction solvent, and the fraction may be obtained by fractionating a 70% ethanol extract of Korean ginseng using methanol as a fractionation solvent.

When separation and purification are performed using the extraction solvent and the fractionation solvent, it is possible to separate and purify non-saponin compounds, and to identify an active ingredient, which is contained only in Korea ginseng cultivars and is not contained in other ginseng species, among the non-saponin compounds. Thus, this active ingredient may be used as a marker for identifying Korean ginseng.

A method of identifying Korean ginseng cultivars using the marker according to another embodiment of the present disclosure includes of: 1) separating ginseng species samples from different ginsengs groups whose species are unidentifiable; 2) preparing ginseng extracts by powdering each of the separated ginseng species samples, followed by extraction using an extraction solvent; 3) obtaining fractions by fractionation of the ginseng extracts; and 4) measuring the UPLC spectrum of the fraction for each ginseng species using a PAD detector at a UV wavelength of 254 nm, wherein ginseng containing an active ingredient showing a first peak at 13.3 min and a second peak at 13.5 min in the results of measuring the UPLC spectrum is Korean ginseng (Panax ginseng C.A. Meyer).

As described above, various ginseng species are distributed in the global market, and representative examples thereof include Korean ginseng (Panax ginseng CA Meyer), American ginseng (Panax quinquefolium) and Chinese ginseng (Panax notoginseng).

There is no difference in appearance between the ginseng species, and it is not easy to distinguish between these ginseng species. If these ginseng species are distributed indiscriminately in the market, a problem may arise in that cheap Chinese ginseng passed off as Korean ginseng is sold.

Therefore, when the method for identifying Korean ginseng cultivars according to the present disclosure is used, it is possible to easily check whether different ginseng species are Korean ginseng by determining whether the active ingredient is contained in fractions from some samples, and it is possible to identify Korean ginseng cultivars with very high accuracy.

The present disclosure provides a marker for identification of Korean ginseng cultivars, which is obtained by identifying and separating an active ingredient, which is contained only in Korean ginseng cultivars, among non-saponin components. In addition, the present disclosure makes it possible to determine whether a product sold as Korean ginseng is Korean ginseng by separating and purifying the active ingredient without using a conventional molecular biological method.

### BRIEF DESCRIPTION OF THE DRAWINGS

FIG. 1 shows the process of extracting ginseng and fractionating the extract according to one embodiment of the present disclosure.

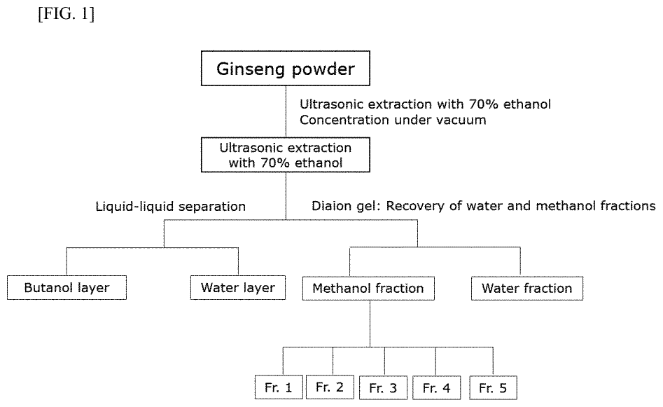

FIG. 2(a), FIG. 2(b), FIG. 2(c) and FIG. 2(d) show the results of performing TLC analysis of extracts from various ginseng species according to one example of the present disclosure.

FIG. 3(a), FIG.3(b), FIG. 3(c), FIG. 3(d), FIG. 3 (e) and FIG. 3(f) show the results of performing TLC analysis of fractions from various ginseng species according to one example of the present disclosure.

FIG. 4 shows a UPLC chromatogram of a ginseng fraction according to one example of the present disclosure.

FIG. 5 shows MS/MS data for specific peak values on the UPLC chromatogram of the ginseng fraction according to one example of the present disclosure.

### DESCRIPTION OF SPECIFIC EMBODIMENTS

Hereinafter, examples of the present disclosure will be described in detail so that those of ordinary skill in the art can easily carry out the present disclosure. However, the present disclosure may be embodied in a variety of different forms and is not limited to the examples described herein.

Ginseng Sample Preparation

Various ginseng species that that are generally distributed in the world market were selected. Specifically, a total of 12 ginseng species, cultivated in different areas and having different ages, were selected. They include Panax ginseng C.A. Meyer (called Korean ginseng), Panax quinquefolium (called American ginseng), and Panax notoginseng (called Chinese ginseng). Before the use of the ginseng species in the experiment, all the root parts, including the main root, the lateral root and the fine root, were powdered and stored. The selected ginseng species are as follows.

\textcircled{1} K-28 (Korean ginseng/Yeoju, Korea/5 years old)

\textcircled{2} (K-29 (Korean ginseng/Buyeo, Korea/4 years old)

\textcircled{3} K-43 (Korean ginseng/Cheongju, Korea/6 years old)

\textcircled{4} K-44 (Korean ginseng/Keumsan, Korea/4 years old)

\textcircled{5} G-3 (Korean ginseng/Changbai mountain/6 years old)

\textcircled{6} H-3 (American ginseng/Hong Kong/4 years old)

\textcircled{7} H-5 (American ginseng/Changbai mountain/4 years old)

\textcircled{8} H-6 (American ginseng/Changbai mountain/5 years old)

\textcircled{9} H-7 (American ginseng/Changbai mountain/6 years old)

[symbol] J-1 (Chinese ginseng/Changbai mountain/4 years old)

[symbol] J-3 (Chinese ginseng/Changbai mountain/6 years old)

[symbol] S-4 (Chinese ginseng/Sichuan, China/4 years old)

Experimental Method

Ginseng Extraction and Separation

1. Reagents and Instruments

For sample extraction and separation, ethanol, methanol and water were used, and solvents (manufactured by Samchun Pure Chemical Co.) of grade 1 were used. For separation, Diaion HP-20 and MCI CHP 20P (Supelco, St. Louis, Mo., USA) were used as gels.

2. Extraction

70 g of powder for each sample was accurately weighed, and then 1,000 mL of 70% ethanol was added thereto. Each of the solutions was subjected to ultrasonic extraction twice for 1 hour and 30 minutes, and each extract was concentrated and filtered.

3.1. Liquid-Liquid Separation

Before concentration of the 70% ethanol extract for each sample, only ethanol was evaporated using a rotary evaporator (N-1100V, Eyela, Tokyo, Japan). Thereafter, n-butanol in the same volume as the remaining water was added, followed by vortexing for 1 minute to cause mass transfer between the solvents. For separation by the density difference between the two solvents, centrifugation was performed at 4,000×g for 10 minutes. The two separate solvents were recovered separately, and powder was recovered from the butanol layer by evaporating the solvent using a rotary evaporator, and powder was recovered from the water layer by subliming water using a freeze dryer (FDS8508, Ilshinbiobase, Korea).

3.2. Separation by Column Chromatography

3.2.1. Diaion HP-20 Column Chromatography

The concentrate of the 70% ethanol extract for each sample was dissolved in water and then loaded onto a column packed with Diaion HP-20, a synthetic adsorbent. Diaion HP-20 is a copolymer of styrene and divinyl benzene (DVB), and is characterized in that it has a large number of pores distributed therein, and thus it has a relatively large surface area and a substance adsorbed into the pores is easily eluted. Since Diaion HP-20 has hydrophobicity, hydrophobic groups such as organic molecules are adsorbed thereto. Thus, in this experiment, Diaion HP-20 was used to remove hydrophilic substances such as sugars because these hydrophilic substances are not adsorbed thereto. Thereafter, the adsorbed substances were eluted with methanol, collected, and then concentrated.

The amount of the methanol fraction for each sample is as follows: K-28 (3.0125 g), K-29 (1.2000 g), K-43 (2.5706 g), K-44 (2.1026 g), G-3 (1.7450 g), H-3 (2.5003 g), H-5 (3.2654 g), H-6 (3.9624 g), H-7 (3.0825 g), J-1 (5.9016 g), J-3 (1.4224 g), and S-4 (5.4512 g).

3.2.2. MCI GEL CHP 20P Column Chromatography

Each concentrated methanol fraction was dissolved in 30% methanol and then loaded onto a column packed with MCI CHP 20P, a synthetic adsorbent. MCI GEL CHP 20P is also a copolymer of styrene and DVB, and can separate substances having a small size because the particle size and pore size thereof are smaller than those of Diaion HP-20. After loading, elution was performed by injecting a solvent in the order of a 30% methanol fraction, a 60% methanol fraction and a 100% methanol fraction, and as a result, substances were separated according to the solvent affinity.

The amount of the MCI fraction for each sample is as follows:

\textcircled{1} K-28: MCI-1 (0.2708 g), MCI-2 (0.0452 g), MCI-3 (0.1092 g), MCI-4 (0.2735 g), MCI-5 (0.2201 g), MCI-6 (0.5141 g), and MCI-Wash (1.087 g)

\textcircled{2} K-29: MCI-1 (0.0146 g), MCI-2 (0.1343 g), MCI-3 (0.1475 g), MCI-4 (0.1348 g), MCI-5 (0.0548 g), MCI-6 (0.2488 g), and MCI-Wash (0.4182 g)

\textcircled{3} K-43: MCI-1 (0.0784 g), MCI-2 (0.0724 g), MCI-3 (0.1604 g), MCI-4 (0.3535 g), and MCI-Wash (1.1503 g)

\textcircled{4} K-44: MCI-1 (0.1162 g), MCI-2 (0.2941 g), MCI-3 (0.2708 g), and MCI-Wash (0.501 g)

\textcircled{5} G-3: MCI-1 (0.3813 g), MCI-2 (0.0488 g), MCI-3 (0.0684 g), MCI-4 (0.2324 g), and MCI-Wash (0.6907 g)

\textcircled{6} H-3: MCI-1 (0.1392 g), MCI-2 (0.0143 g), MCI-3 (0.0359 g), MCI-4 (0.0678 g), MCI-5 (0.4783 g), and MCI-Wash (1.4478 g)

\textcircled{7} H-5: MCI-1 (0.1779 g), MCI-2 (0.2503 g), MCI-3 (0.9307 g), and MCI-Wash (1.8201 g)

\textcircled{8} H-6: MCI-1 (0.1284 g), MCI-2 (0.0461 g), MCI-3 (0.0619 g), MCI-4 (0.1454 g), MCI-5 (0.3934 g), MCI-6 (0.971 g), and MCI-Wash (2.3284 g)

\textcircled{9} H-7: MCI-1 (0.1736 g), MCI-2 (0.103 g), MCI-3 (0.4645 g), and MCI-Wash (1.1273 g)

[symbol] J-1: MCI-1 (0.0217 g), MCI-2 (2.5956 g), MCI-3 (0.161 g), MCI-4 (1.0119 g), and MCI-Wash (0.934 g)

[symbol] J-3: MCI-1 (0.1771 g), MCI-2 (0.5237 g), MCI-3 (0.6254 g), MCI-4 (0.06 g), and MCI-Wash (0.0551 g)

[symbol] S-4: MCI-1 (0.1458 g), MCI-2 (0.2718 g), MCI-3 (1.8632 g), MCI-4 (0.1048 g), and MCI-Wash (2.3908 g)

Verification of Necessity of Column Chromatography

1. Reagents and Instruments

Solvents used for development in thin layer chromatography (TLC) were methanol, water and chloroform, which were all of grade 1, and Kieselgel 60 F254 was used as the TLC plate. For staining of the TLC plate, 10% H₂SO₄, FeCl₃, Dragendorffs reagent and Ninhydrin's reagent were used.

2. Experimental Method

One species was selected from each of Korean ginseng species, American ginseng species and Chinese ginseng species. For each selected species, a 70% ethanol extract, a liquid-liquid separation product and a column chromatography separation product were used and injected in equal amounts. Each sample was loaded onto the TLC plate and developed using chloroform: methanol: water=70:30:4 (v/v/v) as a developing solvent. After completion of the development, the TLC plates were stained with various reagents, and the results were comparatively analyzed.

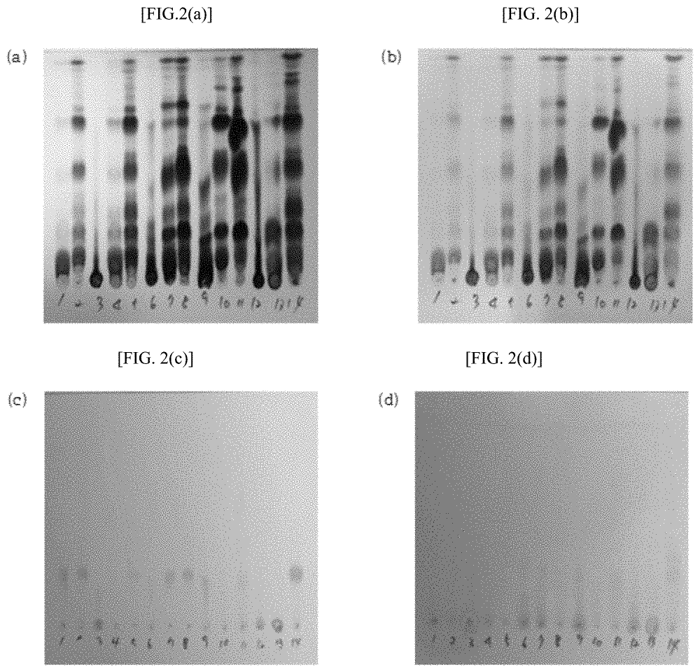

The results are shown in FIG. 2. The numbers in FIG. 2 denote the following extracts and fractions.

1: 70% ethanol extract for K-43

2: butanol layer of liquid-liquid separation product for K-43

3: water layer of liquid-liquid separation product for K-43

4: 70% ethanol extract for G-3

5: butanol layer of liquid-liquid separation product for G-3

6: water layer of liquid-liquid separation product for G-3

7: 70% ethanol extract for H-6

8: butanol layer of liquid-liquid separation product for H-6

9: water layer of liquid-liquid separation product for H-6

10: 70% ethanol extract for 3-13

11: butanol layer of liquid-liquid separation product for J-1

12: water layer of liquid-liquid separation product for J-1

13: water fraction treated with Diaion HP-20 for K-43

14: methanol fraction treated with Diaion HP-20 for K-43

Before comparative analysis for each ginseng sample was performed, it was decided to stain the TLC plate with 10% sulfuric acid because the staining results indicate that staining with 10% sulfuric acid was most obvious and visible. The Examples included in the description of the present disclosure were based on the results in FIG. 2(a) obtained by staining with 10% sulfuric acid. [oo148] First, as a result of comparing the staining results for each ginseng species, it was confirmed that the 70% ethanol extract not subjected to separation (1, 4, 7 and 10 in FIG. 2) was not well developed, suggesting that the process of separation is necessary.

It was shown that the butanol layer of the liquid-liquid separation product for each ginseng species was well developed and also well separated.

As a result of comparing the samples eluted with methanol among the samples, which were obtained from the same ginseng species (K-43) but were subjected to different separation processes, it was confirmed that the methanol fraction (14 in FIG. 2) obtained by treatment with Diaion HP-20 was more visible than the methanol layer (2) of the liquid-liquid separation product. In addition, as a result of examining the degree of darkness of the band, it was confirmed that the methanol fraction (14) was overall darker, and had larger amounts of substances, suggesting that treatment with Diaion HP-20 is a more efficient method in terms of recovery. Based on the above results, a separation method using Diaion HP-20 rather than liquid-liquid separation was selected.

Comparative Analysis of MCI Fractions

1. Reagents and Instruments

Solvents used for development in thin layer chromatography (TLC) were methanol, water and chloroform, which were all of grade 1, and Kieselgel 60 F254 was used as the TLC plate. For staining of the TLC plate, 10% H₂SO₄ was used.

2. Experimental Method

All the MCI fractions from the 12 ginseng species were used as samples. Each sample was loaded onto the TLC plate and developed using chloroform: methanol: water=70:30:4 (v/v/v) as a developing solvent. After completion of the development, the patterns of the TLC plates at a UV wavelength of 254 nm were comparatively analyzed, and then the patterns of the TLC plates stained with 10% sulfuric acid were comparatively analyzed.

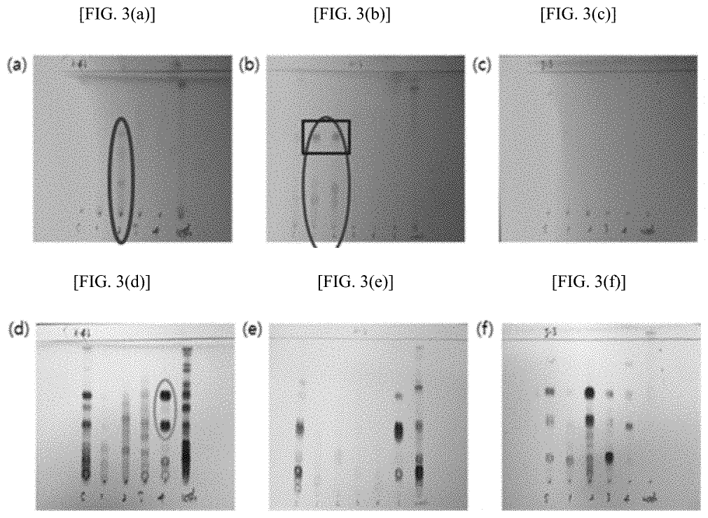

The results of the experiment are shown in FIG. 3. In FIG. 3, (a) is for K-43 (UV 254 nm), (b) is for H-3 (UV 254 nm), (c) is for J-3 (UV 254 nm), (d) is for K-43 (stained with 10% sulfuric acid), (e) is for H-3 (stained with 10% sulfuric acid), and (f) is for J-3 (stained with 10% sulfuric acid).

In the TLC plates of FIGS. 3(a) and 3(b), the portion with a red circle corresponds to a compound which is found commonly found in the ginseng species and presumed to be a phenolic substance.

In FIG. 3(b), the portion marked with a blue square was presumed to be different from the component found in K-43 because it was not stained with 10% sulfuric acid even though it appeared at a similar height in K-43.

In FIG. 3(b), the portion marked with a yellow circle is a portion expected to be ginsenosides.

As a result of the experiment, ginsenosides in ginseng were found in the back part of the MCI fraction, and specific phenolic components in each of Korean ginseng and American ginseng were found in the front part of the MCI fraction.

In addition, the results of analysis at a UV wavelength of 254 nm and the results of staining with 10% sulfuric acid indicated that the phenolic components in Korean ginseng differ from the phenolic components in American ginseng. Thus, the phenolic components in Korean ginseng, which differ from those in other ginseng species, were analyzed.

HPLC Analysis

1. Reagents and Instruments

For analysis, acetonitrile (HPLC grade, J. T. Baker, Philipsbug, N.J., USA), distilled water (HPLC grade), and formic acid (ACS grade, Milipore, Finland) were used. As a HPLC apparatus, Agilent 1260 infinity II (Waldbronn, Germany) was used, and as a column, CAPCELL PAK C18 MG S5 (Shiseido, 5 μm, 4.6×250 mm, Tokyo, Japan) was used.

2. Analysis Conditions

A mixture of 0.1% (v/v) formic acid with D.W and ACN was used in gradient elution mode. The proportion of ACN was increased sequentially from 5% ACN at 0 minutes to 30% ACN at 30 minutes and 60% ACN at 40 minutes, and the column was washed with 95% ACN at 45 minutes, 95% ACN at 50 minutes, 5% ACN at 52 minutes and 5% ACN at 55 minutes. The development temperature was 30° C., the flow rate was set to 0.6 mL/min, and the injection volume of the sample was 10 μL. 10 mg of the sample was mixed with 1.0 mL of 50% methanol to a concentration of 10 mg/mL, and the sample solution was filtered using a 0.45 μm syringe filter made of PTFE. The detection of substances was performed by measuring the absorbance at 254 nm using a PAD detector.

Specifically, in order to select those corresponding to the substances detected in fraction 2 of K-43 at UV 254 nm as confirmed by TLC, the top 10 peaks in the HPLC chromatogram were selected.

Among these peaks, peaks, which are detected in Korean ginseng and not detected in American ginseng and Chinese ginseng, were selected.

As a result of comparing a total of 64 MCI fractions, it was confirmed that only No. 6 peak and No. 7 peak among the 10 candidate peaks satisfied the above-described conditions. These peaks were presumed to be specific non-saponin components that are found only in Korean ginseng. Thereafter, the substances corresponding to No. 6 peak and No. 7 peak were analyzed by UPLC analysis.

UPLC-ESI-Q-TOF-MS Analysis

1. Reagents and Instruments

For analysis, acetonitrile (HPLC grade, J. T. Baker, Philipsbug, N.J., USA), distilled water (HPLC grade), and formic acid (ACS grade, Milipore, Finland) were used. As a UPLC apparatus, Bruker compact (Daltonik, Bremen, Germany) was used, and as a column, ACQUITY UPLC CSH C18 (Waters, 1.7 μm, 2.1×150 mm, Milford, Mass., USA) was used.

2. Analysis Conditions

A mixture of 0.1% (v/v) formic acid with D.W and ACN was used in gradient elution mode. The proportion of ACN was increased from 5% ACN at 0 minutes to 20% ACN, and the column was washed with 95% ACN at 22 minutes, 95% ACN at 27 minutes, 5% ACN at 29 minutes and 5% ACN at 35 minutes. The development temperature was 30° C., the flow rate was set to 0.2 mL/min, and the injection volume of the sample was 10 μL. 10 mg of the sample was mixed with 1.0 mL of 50% methanol to a concentration of 10 mg/mL, and the sample solution was filtered using a 0.45 μm syringe filter made of PTFE. The detection of substances was performed by measuring the absorbance at 254 nm using a PAD detector.

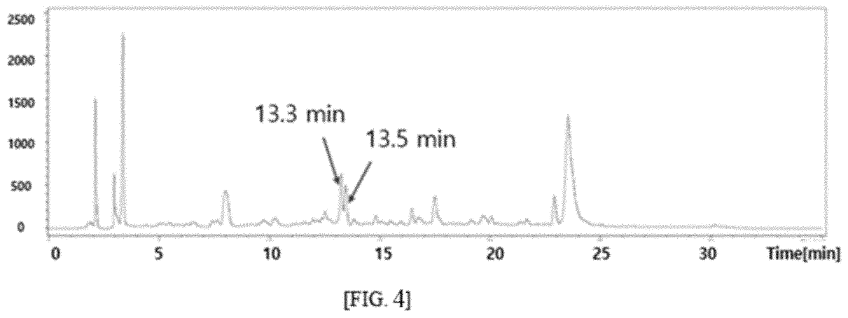

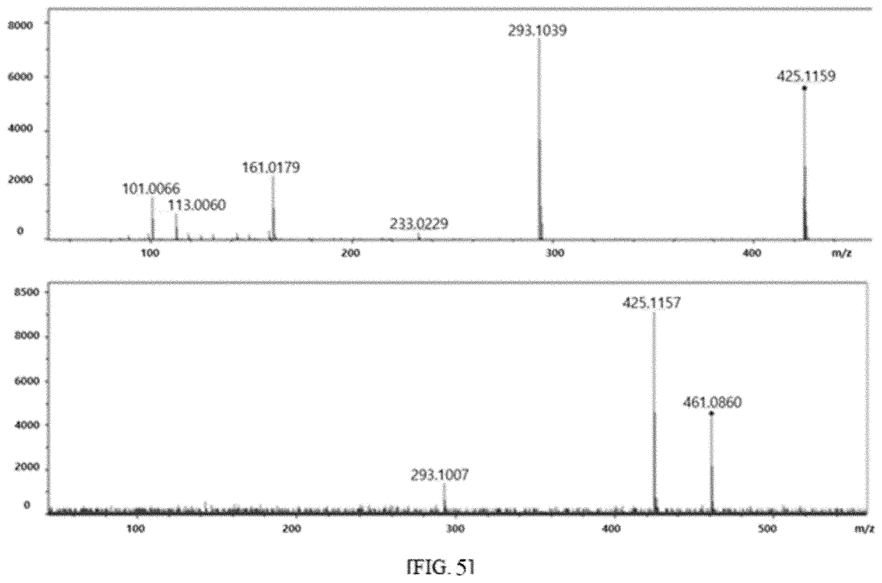

The experimental results are shown in FIGS. 4 and 5.

Specifically, it was confirmed that, even in the UPLC chromatogram based on the UV spectrum, No. 6 peak detected on the HPLC chromatogram was detected at 13.3 min, and No. 7 peak was detected at 13.5 min.

The m/z value of the peak detected at 13.3 min on the UPLC chromatogram was 425.1159, and thus the peak was expected to correspond to a substance having a molar mass of about 426.12 g/mol. MS/MS analysis indicated that an expected material showing this pattern is lasiocarpin A.

The m/z value of the peak detected at 13.5 min on the UPLC chromatogram was 461.0860, and thus the peak was expected to correspond to a substance having a molar mass of about 462.09 g/mol. MS/MS analysis indicated that an expected material showing this pattern is luteolin-7-glucuronide.

The results of the analysis are shown in Table 1 below.

TABLE 1

| Retention | Substance | Molar | |
|---|---|---|---|
| time | name | mass | Structure |
| 13.3 min | Lasiocarpin A | 426.4 g/mol | 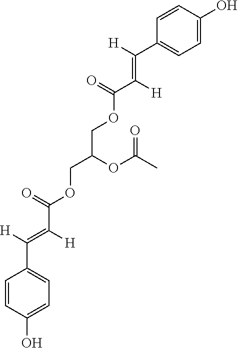 [SMILES: CC(=O)OC(COC(=O)/C=C/c1ccc(O)cc1)COC(=O)/C=C/c1ccc(O)cc1] |
| 13.5 min | Luteolin-7- glucuronide | 462.4 g/mol | 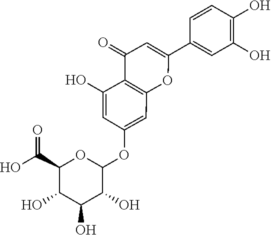 [SMILES: O=C(O)[C@H]1OC(Oc2cc(O)c3c(=O)cc(-c4ccc(O)c(O)c4)oc3c2)[C@H](O)[C@@H](O)[C@@H]1O] |

As a result of examining whether the peaks at 13.3 min and 13.5 min wound also be found in the 70% ethanol extracts in addition to the MCI fractions, it was confirmed that the peaks were found in Korean ginseng, but did not appear in American ginseng and Chinese ginseng.

Table 2 below summarizes the ppm values of the peaks detected in the Korean ginseng.

TABLE 2

| Ginseng species | ppm of peak at 13.3 min | ppm of peak at 13.5 min |
|---|---|---|
| K-8 | 0.03 | 0.06 |
| K-13 | 0.07 | 0.06 |
| K-16 | 0.08 | 0.05 |
| K-18 | 0.20 | 0.06 |
| K-19 | 0.14 | 0.30 |
| K-20 | 0.00 | 0.00 |
| K-21 | 0.19 | 0.06 |
| K-23 | 0.27 | 0.11 |
| K-26 | 0.28 | 0.08 |
| K-27 | 0.22 | 0.09 |
| K-28 | 0.14 | 0.07 |
| K-29 | 0.28 | 0.12 |
| K-30 | 0.14 | 0.08 |
| K-32 | 0.11 | 0.05 |
| K-33 | 0.39 | 0.11 |
| K-34 | 0.14 | 0.05 |
| K-35 | 0.12 | 0.06 |
| K-36 | 0.13 | 0.08 |
| K-37 | 0.10 | 0.11 |
| K-38 | 0.11 | 0.06 |
| K-39 | 0.11 | 0.07 |
| K-40 | 0.23 | 0.09 |
| K-42 | 0.27 | 0.13 |
| K-42 | 0.23 | 0.11 |
| K-43 | 0.18 | 0.10 |
| K-44 | 0.24 | 0.10 |
| K-45 | 0.12 | 0.06 |
| K-46 | 0.13 | 0.05 |
| K-47 | 0.20 | 0.11 |
| K-48 | 0.19 | 0.07 |
| K-49 | 0.27 | 0.08 |
| K-50 | 0.20 | 0.07 |
| K-51 | 0.19 | 0.06 |
| K-52 | 0.19 | 0.16 |
| G-1 | 0.13 | 0.07 |
| G-2 | 0.00 | 0.00 |
| G-3 | 0.00 | 0.00 |
| G-5 | 0.25 | 0.08 |
| G-6 | 0.15 | 0.06 |
| G-9 | 0.18 | 0.04 |
| G-10 | 0.12 | 0.05 |
| G-13 | 0.21 | 0.05 |
| G-14 | 0.17 | 0.05 |

From the above experimental results, it can be confirmed that Korean ginseng species contain a specific component as a non-saponin compound, unlike other ginseng species. The specific compound identified based on the above experimental results may be used as a marker for identifying Korean ginseng cultivars.

Although the preferred embodiments of the present disclosure have been described in detail above, the scope of the present disclosure is not limited thereto, and various modified and improved forms made by those skilled in the art on the basis of the basic concept of the present disclosure defined in the appended claims also fall within the scope of the present disclosure.

What is claimed is:

1. A marker for identifying Korean ginseng cultivars, which is an active ingredient contained in a fraction separated from an extract of Korean ginseng (Panax ginseng C.A. Meyer),
wherein the active ingredient is a non-saponin component, and
the active ingredient shows a first peak at 13.3 min and a second peak at 13.5 min when a UPLC spectrum of the fraction is measured using a PAD detector at a UV wavelength of 254 nm.

2. The marker of claim 1, wherein the active ingredient is a compound represented by the following Formula 1: 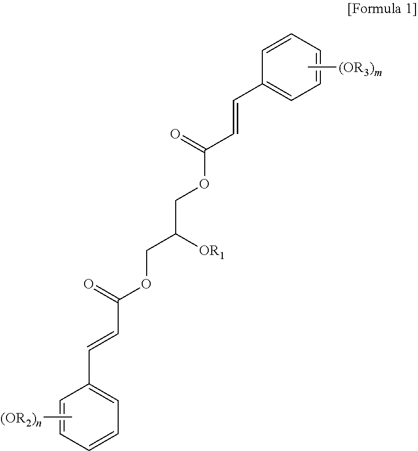
wherein
n and m are the same as or different from each other and are each independently an integer ranging from 1 to 5, and
R₁ to R₃ are the same as or different from one another and are each independently selected from the group consisting of hydrogen, hydrogen, deuterium, a ketone group, an alkyl group having 1 to 30 carbon atoms, and an alkenyl group having 2 to 10 carbon atoms.

3. The marker of claim 2, wherein the active ingredient has a molar mass of 410 to 430 g/mol.

4. The marker of claim 1, wherein the active ingredient is a compound represented by the following Formula 2: 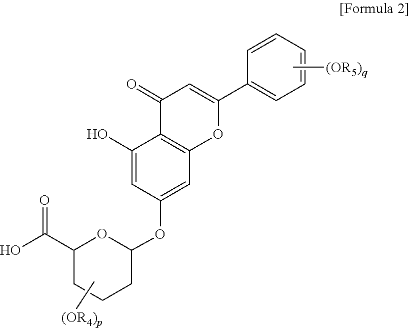
wherein
p is an integer ranging from 1 to 3,
q is an integer ranging from 1 to 5, and
R₄ and R₅ are the same as or different from each other and are each independently selected from the group consisting of hydrogen, deuterium, a ketone group, an alkyl group having 1 to 30 carbon atoms, and an alkenyl group having 2 to 10 carbon atoms.

5. The marker of claim 3, wherein the active ingredient has a molar mass of 455 to 465 g/mol.

6. The marker of claim 1, wherein the extract of Korean ginseng is obtained by extraction using 70% ethanol as an extraction solvent.

7. The marker of claim 1, wherein the fraction is obtained by fractionating a 70% ethanol extract of Korean ginseng using methanol as a fractionation solvent.

8. A method of identifying Korean ginseng cultivars using the marker, the method comprising steps of:
1) separating ginseng species samples from different ginseng groups whose species are unidentifiable;
2) preparing ginseng extracts by powdering each of the separated ginseng species samples, followed by extraction using an extraction solvent;
3) obtaining fractions by fractionation of the ginseng extracts; and
4) measuring a UPLC spectrum of the fraction for each ginseng species using a PAD detector at a UV wavelength of 254 nm,
wherein ginseng containing an active ingredient showing a first peak at 13.3 min and a second peak at 13.5 min in the results of measuring the UPLC spectrum is Korean ginseng (Panax ginseng C.A. Meyer).
